(19) United States
(12) Patent Application Publication
(10) Pub. No.: US 2018/0134453 A1
(43) Pub. Date: May 17, 2018

(54) Inner Cover of a Plastic Material for use as a Sterile Cover for the Inside of a Rectangular Plastic Container, an Assembly Including the Inner Cover and a Method for Making a Sterile Rectangular Plastic Container

(71) Applicant: Wassenburg Medical B.V., Dodewaard (NL)

(72) Inventor: Ronald Wassenburg, Dodewaard (NL)

(73) Assignee: Wassenburg Medical B.V., Dodewaard (NL)

(21) Appl. No.: 15/712,831

(22) Filed: Sep. 22, 2017

(30) Foreign Application Priority Data
Sep. 23, 2016 (NL) NL2017520

(51) Int. Cl. B65D 25/14; B65D 1/34; B65D 1/40; B65D 43/02; B65D 51/24; B08B 1/00; A61B 50/30

(52) U.S. Cl. CPC B65D 25/14; B65D 1/34; B65D 1/40; A61B 50/30; B65D 51/245; B08B 1/006; B65D 43/0202

(57) ABSTRACT

Inner sleeve made of plastic material for use as a sterile cover for the inside of a rectangular transportation tray having a bottom and upright sides, which inner sleeve has a rectangular basic shape with a surface similar to the bottom surface of the transportation tray, wherein corner portions of the inner sleeve are provided with eyelets which can be hooked behind hooks of the transportation tray, in such a way that the inner sleeve covers the inner side of the transportation tray in a sterile manner, wherein the sides of the rectangular basic shape are provided with outwardly extending lateral flaps, wherein each corner portion which is provided with an eyelet is also provided with a tongue part which extends outwardly from the eyelet, wherein the tongue part is of such dimensions, that the tongue part can be folded over a hook of the transportation tray in a covering manner.

Method for preparing a rectangular transportation tray having a bottom and upright sides with a top edge in a sterile manner, which transportation tray is suitable for transporting medical equipment, in particular endoscopic equipment.

The invention relates to an inner sleeve made of plastic material for use as a sterile cover for the inside of a rectangular transportation tray. In addition, the invention relates to an assembly of the inner sleeve with other products, and to a method for preparing a rectangular transportation tray in a sterile manner using such an inner sleeve.

An inner sleeve according to the above type is used, inter alia, for transportation trays which are suitable for transporting medical equipment, more particularly endoscopic equipment.

After use, medical equipment in hospitals is transported to a central collecting location for cleaning the equipment in a transportation tray. Subsequently, the transportation tray used is made ready for transporting sterile equipment from a central dispensing location to the location where the medical operation is to be performed. In hospitals, transportation trays are regularly cleaned, but not for each transport as this would take too much time. In addition, cleaning in itself does not always produce a result which is sufficiently sterile by clinical standards. Therefore, inner sleeves have been developed which cover the inside of a transportation tray in a sterile manner. The inner sleeves are used once for transportation and are mounted under aseptic conditions by a user wearing gloves. Mounting an inner sleeve in the tray is necessary to prevent that it starts sliding during transportation. This sliding is undesirable because of the risk that the inside of the transportation tray will no longer be fully covered and the sterile equipment may become contaminated. Then, the sterile equipment is placed on the inner sleeve which has been mounted in the tray and, finally, a sterile outer sleeve is preferably mounted over the outer edge to cover the top side of the tray.

In the field, transportation trays which have an outwardly curved top edge which continues far downwards are common. Such a type of transportation tray is produced, for example, by Medicart International Ltd. This company is also the holder of US patent U.S. Pat. No. 6,749,063, the figures of which show such an embodiment of a tray having a curved top edge.

However, cleaning a transportation tray of this known type in dishwashers has drawbacks: often, water remains behind on the inner side of the curved outer edge, thus preventing proper drying. The risk of sources of contamination remaining behind or being produced on this inner side is thus increased. At the same time, an inner sleeve is used for such a transportation tray which is pulled over the entire outer edge and is hooked behind the curved outer edge. This bears the risk that the user directly touches a non-sterile area on the inner side of the curved outer edge with his fingertips. Since the user places the equipment in the tray after this operation, there is a risk of contamination.

In addition, the production of this transportation tray by injection-molding is slightly complicated due to the presence of the curved outer edge.

Thus, a new transportation tray has been developed which has a bottom and upright sides with a top edge, in which every corner of the transportation tray is provided with a hook which is formed by two indentations in the top edge of the upright walls on either side of the respective corner. It is possible to mount an inner sleeve behind the hooks so that it remains in place, without requiring a curved outer edge. Due to the absence of a curved outer edge, a sterile end result can more readily be ensured during cleaning of the tray. In addition, this relatively simple design is also easier to manufacture.

With regard to an inner sleeve which fits this new transportation tray, the applicant then examined in which way this could be made sufficiently suitable to obtain a result which is at least on a par and preferably better than is the case with the customary transportation tray with curved outer edge as described above.

It is therefore the object of the invention to provide an inner sleeve which is sufficiently suitable for a transportation tray whose corners are provided with hooks, and which achieves a result which is on a par with or better than the prior art.

To that end, according to a first aspect, the invention provides:

an inner sleeve made of plastic material for use as a sterile cover for the inside of a rectangular transportation tray having a bottom and upright sides,

which inner sleeve has a rectangular basic shape with a surface similar to the bottom surface of the transportation tray, wherein corner portions of the inner sleeve are provided with eyelets which can be hooked behind hooks of the transportation tray in such a way that the inner sleeve covers the inner side of the transportation tray in a sterile manner,

wherein the sides of the rectangular basic shape are provided with outwardly extending lateral flaps, and

wherein each corner portion which is provided with an eyelet is also provided with a tongue part which extends outwardly from the eyelet, wherein the tongue part is of such dimensions, that the tongue part can be folded over a hook of the transportation tray in a covering manner.

When such an inner sleeve is used, a satisfactory end result is achieved, due to the fact that:
the inner sleeve is held in place;
the lateral flaps hang like curtains over the outside of the upright sides of the transportation tray, so that a user does not come into direct contact with the transportation tray when he touches the tray;
the folded tongue parts cover the hooks of the transportation tray, so that direct contact with the—possibly non-sterile—hooks is prevented.

An additional advantage is the fact that the risk of contamination related to the curved outer edge of the prior-art transportation tray no longer presents itself.

It is advantageous if, with the inner sleeve according to the invention, the lateral flap extends outwardly up to at least a length which corresponds to the height of the upright sides of the transportation tray. In practice, such a length results in the flaps covering the sides of the transportation tray at the locations where the user touches the tray with his hands.

Particularly advantageously, the inner sleeve according to the invention is configured in such a way that a substantial part of the respective side and at least the central part of the side is provided with a lateral flap. This is advantageous, because the central part of the side is the area where the user touches the tray in order to hold it in a stable position.

In a particular embodiment of the inner sleeve according to the invention, the lateral flap extends outwardly up to a length which, near the corner portions, is smaller than a length corresponding to the height of the upright sides of the transportation tray. Thus, a saving in material is achieved at locations where, in practice, the length of the lateral flap is less critical.

Preferably, with the inner sleeve according to the invention, the tongue part is wider than the hook of the transportation tray, and the tongue part extends outwardly from the respective eyelet along a length which is at least three times the height of the hook. This results in the tongue part being sufficiently large to adequately cover the inner side of the hook in the folded position.

It is particularly preferred, with the inner sleeve according to the invention, for each eyelet to be configured as a widened hole, preferably having a width which corresponds to the smallest width of the hook of the transportation tray. Such a shape of the eyelet is very suitable to hook behind a hook of the tray. In addition, such a shape of the eyelet corresponds best to the periphery of the hook when the inner sleeve is hooked on.

With the inner sleeve according to the invention, it is preferable if at least two diagonally opposite corner portions are provided with eyelets, and preferably four corner portions are provided with eyelets. This results in the inner sleeve being mounted in such a way that the risk of sliding inside the tray is minimized, thus producing a reliable sterile cover.

With the inner sleeve according to the invention, it is furthermore preferable if each corner portion is pre-formed in a three-dimensional way, for example by welding areas of the plastic material together. In this way, it is possible to produce the inner sleeve from a sheet of plastic material and give it a three-dimensional shape at the corner portions which copies the corners on the inner side of the transportation tray to some degree. This facilitates placing the inner sleeve in the tray and ensures that the inner sleeve fits well into the corners of the tray at the location of the corner portions. Welding the plastic material together is a suitable technique for creating a third dimension in a flat sheet of plastic material.

With the inner sleeve according to the invention, it is particularly preferred if each tongue part is composed of areas of the plastic material which have been welded together. If, for example, the corner portion is preformed in a three-dimensional way by welding the plastic material together, then it is advantageous to also use this technique to form the tongue part.

The inner sleeve according to the invention is produced by cutting a suitable form from a sheet of plastic material, wherein the corner portions may optionally be made from two parts which are welded together subsequently. The plastic material used may be, for example, polyethylene (PE), more specifically high-density (HDPE) or low-density (LDPE), with HDPE being preferred.

Preferably, the sizing of the inner sleeve is in such a range that it is suitable for a transportation tray having a rectangular outer format of 45×55 cm.

The invention also relates to an inner sleeve according to the first aspect of the invention, having a separate added element in the form of an outer sleeve whose peripheral edge is completely or partly provided with elastic material. The outer sleeve is an element which is separate from the inner sleeve and which is also used separately according to the above-described method, in which, successively, the inner sleeve is arranged in the transportation tray, the equipment is placed in the tray and the outer sleeve is pulled over the tray in order to cover the top of the tray.

In this case, the outer sleeve is preferably provided with a sign on one side which denotes a sterile state and is provided on the other side with a sign which denotes a non-sterile state. Thus, the state of the storage tray is directly visible for the user or for an outsider.

In addition, the invention relates to an inner sleeve according to the first aspect of the invention having a cleaning cloth impregnated with a disinfectant as a separate added element.

A second aspect of the invention relates to an assembly comprising:
an inner sleeve according to the first aspect of the invention, and
a rectangular transportation tray having a bottom and upright sides with a top edge, which transportation tray is suitable for transporting medical equipment, in particular endoscopic equipment,
   wherein each corner of the transportation tray is provided with a hook which is formed by two indentations in the top edge of the upright sides on either side of the respective corner.

The respective advantages of this assembly have already been described above with regard to the first aspect of the invention.

This assembly according to the invention relates both to a collection of the two components as separate elements, and to the two components together as cooperating elements in the position in which the inner sleeve is hooked behind the hooks of the transportation tray.

With the assembly according to the invention, the corners of the transportation tray are preferably rounded. In this way, sharp corners are avoided which could cause undesirable cuts in the inner sleeve, or more specifically in the eyelet during hooking when mounting the inner sleeve.

With the assembly according to the invention, the two indentations forming the hook are furthermore preferably configured in such a way that the hook widens in the direction of the top edge. This ensures that the eyelet which is hooked behind the hook is more securely fixed in its position, thus preventing the inner sleeve from unintentionally sliding off the hook.

With the assembly according to the invention, a lower portion of the outer surface of each upright side is furthermore preferably curved in order to serve as a handle. Although the user is able to grasp the tray in a satisfactory manner on the sides, handles are advantageous as these improve the grip on the tray, in particular with relatively heavy loads or when the tray is to be held by hand for a prolonged period of time.

A third aspect of the invention relates to a method for preparing a rectangular transportation tray having a bottom and upright sides with a top edge in a sterile manner, which transportation tray is suitable for transporting medical equipment, in particular endoscopic equipment,

wherein each corner of the transportation tray is provided with a hook which is formed by two indentations in the top edge of the upright walls on either side of the respective corner, comprising the following steps:
a) arranging a sterile inner sleeve according to the first aspect of the invention on the inside of the transportation tray by hooking the eyelets of the inner sleeve behind the hooks of the transportation tray,
b) folding the tongue parts over the respective hooks of the transportation tray so that they cover the inner side of the hooks.

In this case, the inner side of the hook part is optionally treated with a cleaning cloth impregnated with disinfectant to remove possible sources of contamination prior to step a) or b).

The advantages resulting from this method have already been explained above with regard to the first aspect of the invention.

The invention will be described below in more detail with reference to the attached figures, in which:

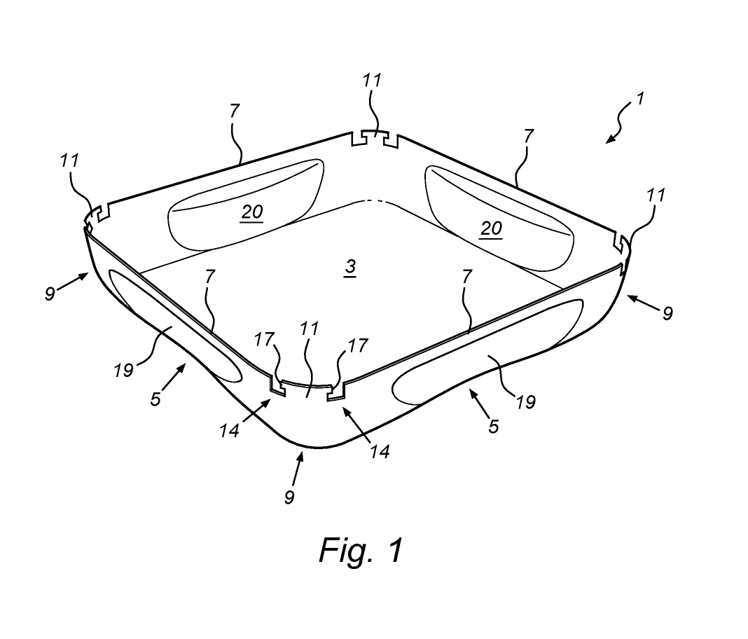

FIG. 1 shows a perspective view of a transportation tray according to a preferred embodiment of the invention;

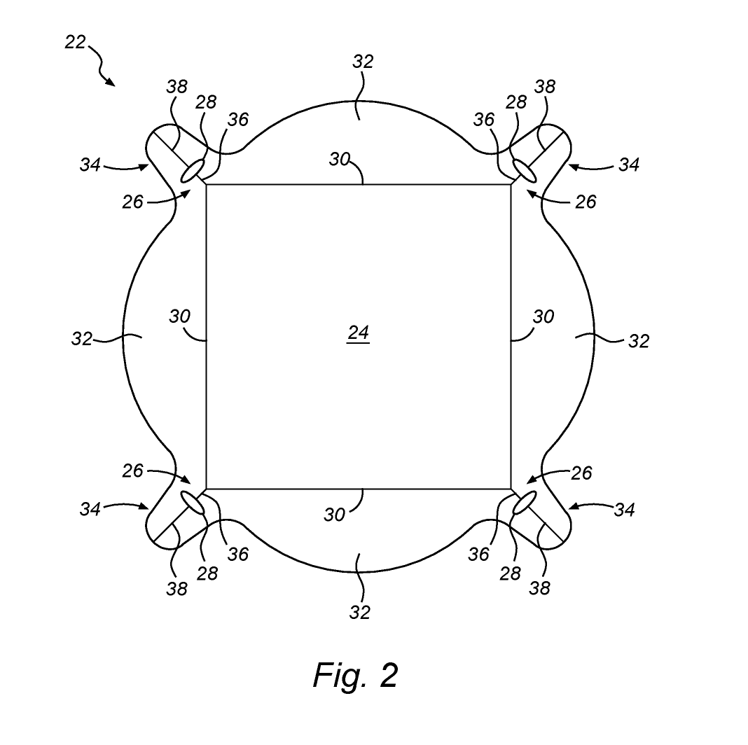

FIG. 2 shows a top view of an inner sleeve according to a preferred embodiment of the invention;

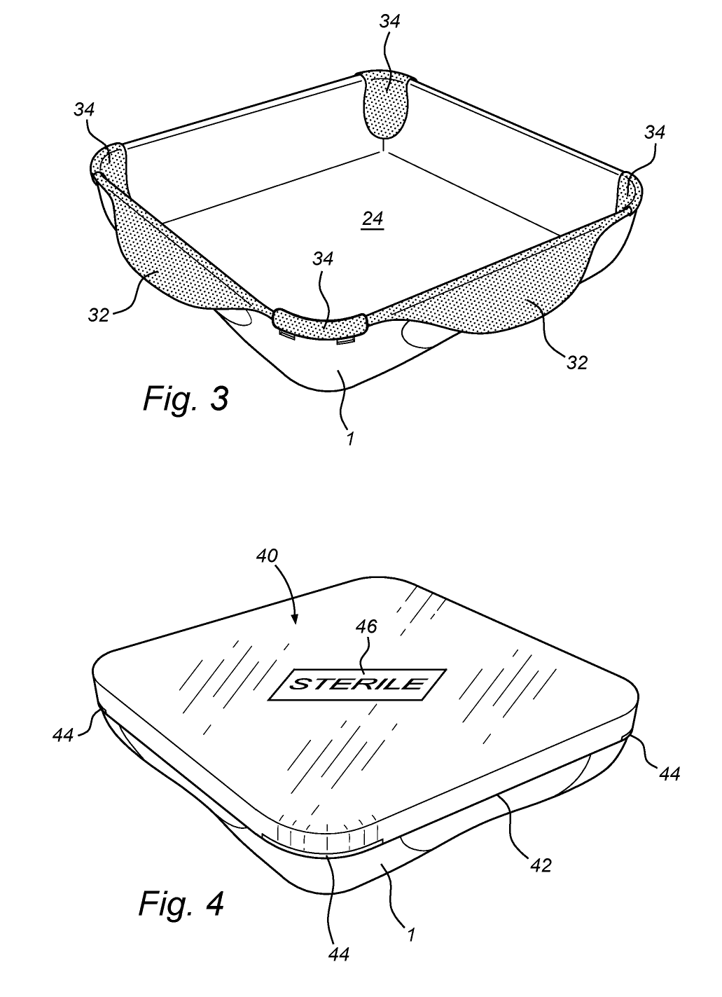

FIG. 3 shows the assembly of a transportation tray with mounted inner sleeve according to a preferred embodiment of the invention;

FIG. 4 shows an assembly of a transportation tray with mounted outer sleeve.

FIG. 1 shows a rectangular transportation tray 1 having a bottom 3 and upright sides 5 with a top edge 7, which transportation tray 1 is suitable for transporting medical equipment, in particular endoscopic equipment, wherein each corner 9 of the transportation tray is provided with a hook 11 which is formed by two indentations 14 in the top edge of the upright sides 5 on either side of the respective corner. The corners 9 of the transportation tray are rounded. The two indentations 14 forming the hook 11 are configured in such a way that the hook has a widening 17 in the direction of the top edge. A lower portion 19 of the outer surface of each upright side 5 is curved in order to serve as a handle. This is visible from the inner side as an indented part 20.

FIG. 2 shows an inner sleeve 22 made of plastic material for use as a sterile cover for the inside of a rectangular transportation tray having a bottom and upright sides, which inner sleeve has a rectangular basic shape 24 with a surface which is similar to the bottom surface 3 of the transportation tray 1, wherein corner portions 26 of the inner sleeve 22 are provided with eyelets 28 which can be hooked behind hooks 11 of the transportation tray. The sides 30 of the rectangular basic shape are provided with outwardly extending lateral flaps 32. Each corner portion 26 is provided with a tongue part 34 which extends outwardly from the eyelet along a length which is approximately four times the height of the hook 11.

The lateral flaps 32 extend outwardly to a length which is slightly greater than the height of the upright sides 5 of the transportation tray 1. Each eyelet 28 is configured as a widened hole having a width which corresponds to the narrowest width of the hook 11 of the transportation tray 1. Each corner portion 26 is preformed in a three-dimensional way by welding together areas of the plastic material, the weld seam 36 of which is visible. A weld seam 38 is also present in the tongue part 34.

FIG. 3 shows the assembly of the inner sleeve 22 from FIG. 2, mounted on the transportation tray 1 from FIG. 1. The lateral flaps 32 which hang over the outer surfaces of the sides of the tray 1 are shown hatched and cover the curved parts which function as handles. The tongue parts 34 which are folded from the outside in over the hooks so that the hooks are covered on the inner and outer side are also shown hatched. The rectangular basic shape 24 covers the bottom of the tray.

FIG. 4 shows the assembly of an outer sleeve 40, mounted on the transportation tray 1 from FIG. 1. The outer sleeve 40 has a peripheral edge 42 whose corners are provided with elastic material 44. On the illustrated side, the outer sleeve is provided with a printed indication 46 of a sterile state.

1. An inner sleeve made of plastic material for use as a sterile cover for the inside of a rectangular transportation tray having a bottom and upright sides,
which inner sleeve has a rectangular basic shape with a surface similar to the bottom surface of the transportation tray, wherein corner portions of the inner sleeve are provided with eyelets which can be hooked behind hooks of the transportation tray in such a way that the inner sleeve covers the inner side of the transportation tray in a sterile manner,
wherein the sides of the rectangular basic shape are provided with outwardly extending lateral flaps,
wherein each corner portion which is provided with an eyelet is also provided with a tongue part which extends outwardly from the eyelet, wherein the tongue part is of such dimensions, that the tongue part can be folded over a hook of the transportation tray in a covering manner.

2. The inner sleeve as claimed in claim 1, wherein the lateral flap extends outwardly up to at least a length which corresponds to the height of the upright sides of the transportation tray.

3. The inner sleeve as claimed in claim 1, wherein a substantial part of the respective side and at least the central part of the side is provided with a lateral flap.

4. The inner sleeve as claimed in claim 1, wherein the tongue part is wider than the hook of the transportation tray, and the tongue part extends outwardly from the respective eyelet along a length which is at least three times the height of the hook.

5. The inner sleeve as claimed in claim 1, wherein each eyelet is configured as a widened hole.

6. The inner sleeve as claimed in in claim 1, wherein at least two diagonally opposite corner portions are provided with eyelets.

7. The inner sleeve as claimed in claim 1, wherein each corner portion is preformed in a three-dimensional way.

8. The inner sleeve as claimed in in claim 8, wherein each tongue part is composed of areas of the plastic material which have been welded together.

9. The inner sleeve as claimed in in claim 1, having a separate added element in the form of an outer sleeve whose peripheral edge is completely or partly provided with elastic material.

10. The inner sleeve as claimed in laim 1, having a cleaning cloth impregnated with a disinfectant as a separate added element.

11. An assembly comprising:
an inner sleeve as claimed in claim 1, and
a rectangular transportation tray having a bottom and upright sides with a top edge, which transportation tray is suitable for transporting medical equipment, in particular endoscopic equipment,
wherein each corner of the transportation tray is provided with a hook which is formed by two indentations in the top edge of the upright sides on either side of the respective corner.

12. The assembly as claimed in claim 11, wherein the corners of the transportation tray are rounded.

13. The assembly as claimed in claim 11, wherein the two indentations forming the hook are configured in such a way that the hook widens in the direction of the top edge.

14. The assembly as claimed in claim 11,
wherein a lower portion of the outer surface of each upright side is curved in order to serve as a handle.

15. A method for preparing a rectangular transportation tray having a bottom and upright sides with a top edge in a sterile manner, which transportation tray is suitable for transporting medical equipment, in particular endoscopic equipment,
wherein each corner of the transportation tray is provided with a hook which is formed by two indentations in the top edge of the upright walls on either side of the respective corner,
comprising the following steps:
a) arranging a sterile inner sleeve as claimed in any of the claims 1-10 on the inside of the transportation tray by hooking the eyelets of the inner sleeve behind the hooks of the transportation tray,
b) folding the tongue parts over the respective hooks of the transportation tray so that they cover the inner side of the hooks.

16. The method as claimed in claim 15, wherein the hook part is treated with a cleaning cloth impregnated with disinfectant prior to step a) or b).

17. The inner sleeve as claimed in claim 5, wherein each eyelet as a width which corresponds to the narrowest width of the hook of the transportation tray.

18. The inner sleeve as claimed in claim 1, wherein four corner portions are provided with eyelets.

19. The inner sleeve as claimed in claim 8, wherein each corner portion is preformed by welding areas of the plastic material together.
